(19) United States
(12) Patent Application Publication
(10) Pub. No.: US 2017/0326338 A1
(43) Pub. Date: Nov. 16, 2017

(54) METHODS AND APPARATUS FOR SECURING A LINE

(71) Applicant: Braidlock Limited, London (GB)

(72) Inventor: Chris WATSON, London (GB)

(21) Appl. No.: 15/529,726

(22) PCT Filed: Nov. 25, 2015
(86) PCT No.: PCT/GB2015/053596

§ 371 Date: May 25, 2017

(30) Foreign Application Priority Data
Nov. 25, 2014 (GB) 1420940.7

(51) Int. Cl. A61M 25/02; B65B 5/04; A61M 39/10; A61B 5/00

(52) U.S. Cl. CPC A61M 25/02; A61M 39/10; B65B 5/04; A61M 2025/0206; A61B 5/00; A61M 2039/1066; A61M 2025/026

(57) ABSTRACT

A fastener for securing a medical or surgical line with respect to a patient, the fastener comprising: an elongate braided tubular sleeve for receiving the line therethrough, the sleeve having a variable length and being capable when lengthened of gripping the line, an attachment arrangement for attaching the sleeve to the patient, and a sensor mounted to the fastener, the sensor being arranged to sense at least one of a physiological parameter of the patient and a parameter in relation to the line.

The present invention relates broadly to the field of fasteners and is particularly concerned with medical or surgical fasteners that secure lines, such as catheters, other types of medical tubing or wires with respect to a patient.

By way of background, catheters are long, thin, flexible tubes of plastics material that can be inserted into a blood vessel or other body cavity for introducing or removing fluids; either liquids or gases. They are used by medical personnel as a matter of routine. In one medical procedure a line or catheter is secured with respect to a patient, for example medical tubing may be secured to a patient's arm, and in another procedure an endotracheal tube may be secured to ventilate a patient. In both cases, it is desirable that, once the line has been inserted, it is securely maintained in position unless a medical practitioner wishes to adjust the positioning of the line.

The insertion of a catheter into a patient is an intricate procedure. Therefore, once a line is in an acceptable position, it is essential that the line is securely anchored to avoid any accidental displacement. The line may need to be in place for a period of weeks: the longer a line remains undisturbed in situ in accordance with planned treatment, the lower the risk of harm to the patient. Similarly, the insertion of an endotracheal tube can be a time-consuming and difficult procedure. Again, it is essential that, once inserted, such tubes are securely fastened in position to avoid potential problems and complications for the patient.

The applicant's earlier application, published as WO-A1-99/10250 describes an improved fastener for securing a line to a patient including a braided sleeve portion for gripping a line having a rigid collar at each end, a flange attached to one of the collars, and an adhesive plaster for securing the flange to a patient. Elongating and narrowing the sleeve applied to the line serves to grip the line and resist movement of the line along its longitudinal axis with respect to the sleeve. The sleeve can be shortened by pushing its ends towards one another. Thus shortened, the sleeve is capable of sliding along the line. A further application of the applicant's, published as WO 2010/049734, also relates to such fasteners.

The present invention provides improved devices for securely maintaining a line in position with respect to a patient.

Embodiments of the disclosure relate to a fastener for securing a medical or surgical line with respect to a patient, the fastener comprising a sensor configured to sense at least one parameter in relation to at least one of the patient, the line and the fastener.

According to the invention, there is provided a fastener for securing a medical or surgical line with respect to a patient, the fastener comprising: an elongate braided tubular sleeve for receiving the line therethrough, the sleeve having a variable length and being capable when lengthened of gripping the line, an attachment arrangement for attaching the sleeve to the patient, and a sensor mounted to the fastener, the sensor being arranged to sense at least one of a parameter in relation to the skin of the patient and a parameter in relation to the line.

Preferably, the sensor is arranged to be adjacent the skin of the patient when the fastener is attached to the patient. The physiological parameter may for example be skin translucence, skin conductance, skin temperature, blood pressure, blood saturation level, or heart rate.

Preferably, the sensor includes a sensor element, a transmitting element such as an antenna and a power source such as a battery. Thus the sensor may be arranged to transmit sensor information wirelessly.

The condition relating to the line may be a parameter of a fluid slow in the surgical line, such as flow rate, fluid temperature, etc. Thus the sensor may for example serve to monitor the integrity of the surgical line.

Preferably, the sleeve has a first end for fixing adjacent to the patient, in use, and a second end for arranging, in use, proximal to medical equipment coupled to the line. The sleeve may be provided with a first substantially rigid collar coupled to the sleeve at the first end of the sleeve and a second substantially rigid collar coupled to the sleeve at the second end of the sleeve, to enable the sleeve to be manipulated to vary the length of the sleeve.

Conveniently, an enlarged flange is attached to the first or second collar, for attaching the fastener to the patient. The a sensor mounted to the flange so as to be positioned adjacent the skin of the patient when the fastener is secured with respect to the patient, the sensor being arranged to sense at least one parameter in relation to the skin of the patient.

The fastener may for example be mounted to the patient using an adhesive plaster secured to the flange, or the flange may be sutured to the patient. The adhesive plaster may be formed of an ‘intelligent’ material, such that the sensor may be incorporated in the adhesive plaster. Thus the fastener may provide information relating to the condition of the patient, such as for example blood pressure or heart rate.

The flange may be attached to the collar by an arm, and the arm may comprise or carry a sensor link between the sensor element and the line. For example, the link may be a wired link, or an optical link.

The sensor may communicate with a monitoring device or system, for example by a wireless link. The fastener may comprise a machine readable marker for pairing the fastener with a patient identification of a patient to whom the fastener is to be attached. Thus the system may provide information relating to a particular patient.

The patient identification may be associated with predetermined sensor parameters for the patient, for example maximum and/or minimum sensor values which may indicate an acceptable range, which may be programmed into the device depending upon the patient's medical condition and the conditions to be monitored or sensed. Thus the system may provide information or alerts appropriate to the patient being monitored. The system may additionally communicate with a device such as a remote or mobile device, which may be associated with or carried by a medical practitioner, such as a mobile phone, which may also be dependent upon or associated with the patient identification.

Thus according to another aspect, the invention provides a system for monitoring a condition relating to a patient, comprising a fastener as defined above, and a monitoring device in communication with the fastener. The system may comprise a plurality of fasteners, and may be arranged to monitor a plurality of patients. For example, the system may be arranged to store information relating to a plurality of patients, and a pairing between each device and a particular patient.

In order that the invention may be more readily understood, reference will now be made to the accompanying drawings, in which:

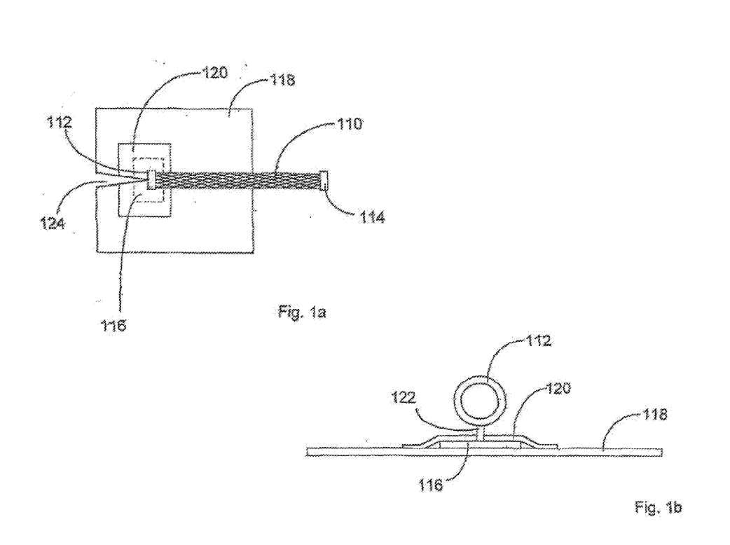

FIGS. 1a and 1b are schematic illustrations of a fastener known from the applicant's previous disclosure, in plan view and a cross-sectional view respectively;

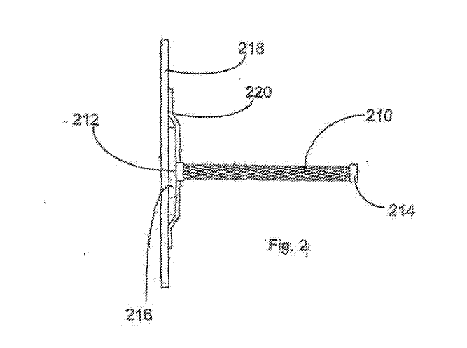

FIG. 2 is a schematic illustrations of a further such fastener in plan view;

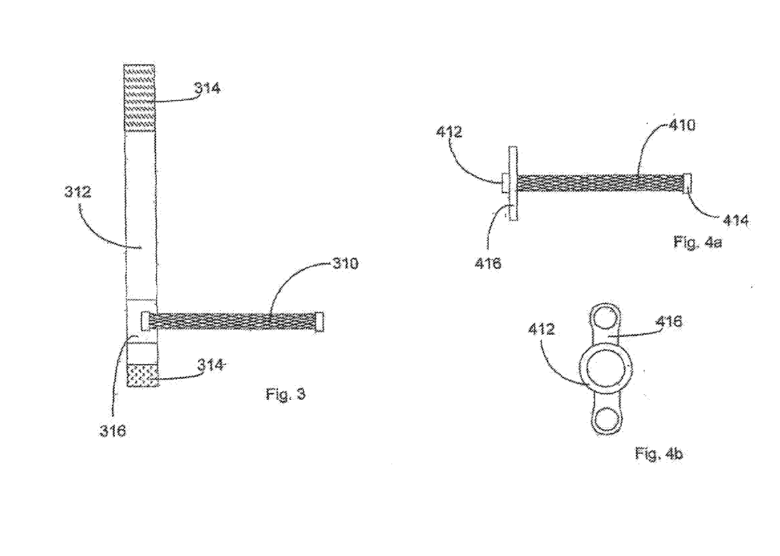

FIG. 3 is a schematic illustration of another such fastener;

FIGS. 4a and 4b are schematic illustrations of yet another such fastener in plan view and a cross-sectional view respectively; and

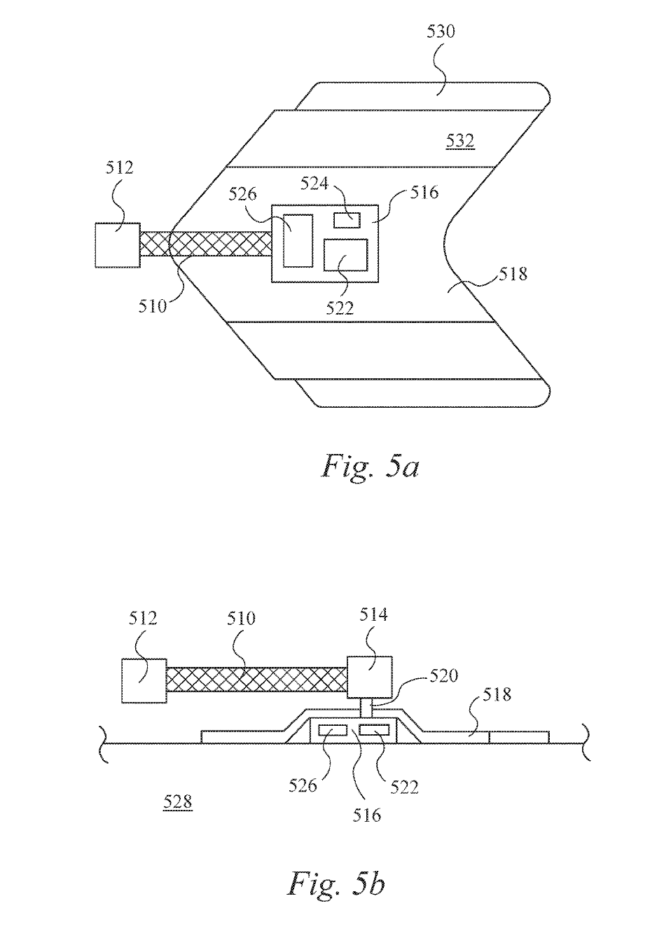

FIGS. 5a and 5b show a plan view and a cross-sectional view, respectively, of a fastener according to an embodiment of the invention;

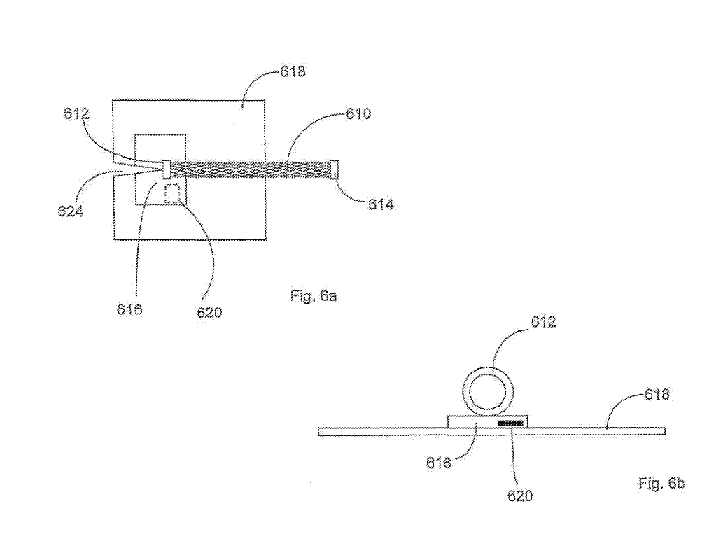

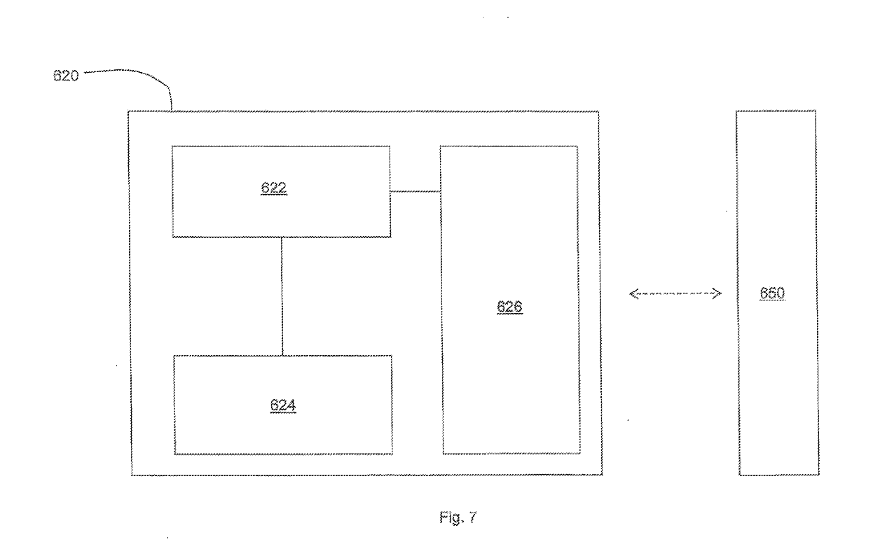

FIGS. 6a and 6b are schematic illustrations of a fastener in plan view and cross-sectional view respectively;

FIG. 7 is a diagram of a system according to another embodiment for use with the fastener; and

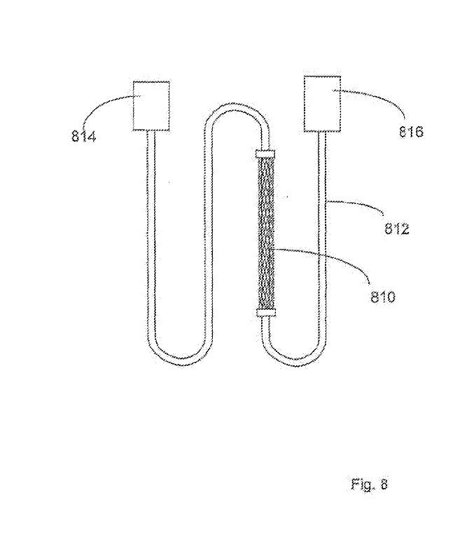

FIG. 8 is a schematic illustration of a giving set including a fastener.

The fastener of FIG. 1a includes a braided tubular sleeve 110, having a collar at each end 112, 114. At one end of the sleeve 110, one of the collars 112 is secured to a flange 116. As illustrated in FIG. 1b, the collar 112 is secured to the flange 116 via an arm portion 122. The arm portion 122 lifts the collar 112 away from the surface of the flange 116, enabling the flange 116 to be secured more easily to the patient.

In this embodiment, the collar 112, the arm 122 and the flange 116 are moulded in one piece. Therefore, the collar 112 is very securely attached to the flange 116.

At each end, the braided sleeve 110 passes inside a collar 112, 114. The strands that make up the sleeve 110 are secured to each of the collars 112, 114 for example using ultrasound welding. Therefore, each strand of the sleeve 110 is securely attached to each collar 112, 114, preventing any fraying or unravelling of the sleeve.

To attach the fastener to a patient, the flange is secured to a plaster 118. The back of the plaster 118 comprises an adhesive material with sufficient adhesion to secure the plaster to a patient's skin. The plaster 118 is provided with backing paper or plastic, which is removed by a medical practitioner when the fastener is in position and the practitioner wishes to secure the fastener to the patient.

The flange 116 is secured to the plaster using a second layer of plaster 120, which is placed over the top of the flange 116 and onto the first plaster layer 118, as illustrated in FIG. 1b. The second layer of plaster 120 extends over the edges of the flange 116 by about 1 cm in each direction to ensure a secure connection of the flange 116 to the first layer of plaster 118.

The first layer of plaster 118 includes a cut-out section 124 which enables a line emerging from the sleeve 110 to have direct access to the patient's skin. The cut-out 124 may take many shapes and forms, for example it may be a hole through which the line can pass. Alternatively, the collar 112 and flange 116 may be arranged at the edge of the plaster 118 so that the line has access to the patient directly without the need for a cut-out section.

In some embodiments, the collar 112 is secured to the arm 122 and the arm 122 is secured to the flange 116 using ultrasound welding technique as described below. The collar 112, the arm 122 and the flange 116 may all be formed from the same material, in this embodiment polyamide.

In alternative embodiment, the arm 122 may be omitted and the collar 112 may be secured directly to the flange 116. In this embodiment, the second adhesive material 120 may still be fitted around the collar 112 and over the flange 116, but it may be more difficult to secure the material 120 to the flange 116 around the collar 112.

As an alternative to the use of the second layer of plaster 120, the flange 116 may be connected to the first plaster layer 118 using welding or heat sealing techniques or another fixing mechanism, such as small rivets, which may also be made from plastic.

An alternative embodiment of the fastener secured by a plaster is illustrated in FIG. 2. This embodiment also includes the braided tubular sleeve 210 with collars 212, 214, one of which is secured or integrally moulded with a flange 216. The flange 216 is secured to a first layer of adhesive plaster 218 by a second layer of adhesive plaster 220 that is placed over the flange 216. However, in contrast to the embodiment shown in FIGS. 1a and 1b, the sleeve of FIG. 2 is held perpendicular to the plaster 218, that is perpendicular to the skin of a patient to which the plaster is secured.

Further embodiments of the fastener are illustrated in FIGS. 3, 4a and 4b. In the embodiment of FIG. 3, the sleeve 310 is attached to a strap 312, which includes a hoop-and-loop portion at each end 314 to enable the strap 312 to be secured at a selected width, for example around a patient's wrist. In order to secure the sleeve 310 to the strap 312, a flange 316 is supplied, which is securely attached to the collar of the sleeve 310. The flange 316 is secured to the strap 312 using an adhesive plaster as described above (not shown) or for example by ultrasound or heat welding the flange to the strap or by other fixing means.

The embodiment illustrated in FIGS. 4a and 4b includes a flange 416 secured around the sleeve 410 behind one of the collars 412, 414. The flange of FIGS. 4a and 4b is preferably secured to the collar using ultrasound welding. As illustrated in FIG. 4b, the flange includes at least one preformed aperture, preferably two, which may be used to secure the fastener to a patient, for example by suturing the fastener to the patient, the sutures being passed through the apertures in the flange 416.

It will be appreciated that, due to the braided construction of the sleeve, the length of the sleeve can be varied by the application of a compression or tension force along the longitudinal axis of the sleeve. It will further be appreciated that elongation causes the sleeve to narrow, whereas shortening the sleeve makes it wider.

Shortening of the sleeve causes the filaments of the braid to bunch together and to lie at a large angle with respect to the longitudinal axis of the sleeve. Therefore, when the sleeve is shortened, the diameter of the aperture formed by the sleeve is at a maximum. In contrast, elongation of the sleeve causes the filaments of the braid to lie at a smaller angle with respect to the longitudinal axis of the sleeve. Hence, the diameter of the aperture formed by the sleeve is at a minimum.

The sleeve is biased to a slightly elongated position so that the diameter of the braided sleeve is slightly narrower than the diameter of the aperture at each end formed by the collars. Therefore, when a tube or line having a similar size diameter to that of the aperture formed by a collar is inserted through the sleeve, the sleeve is biased to grip the tube or line.

It will be appreciated that, once the initial grip of the sleeve around the line is established, further attempts to move the line axially with respect to the sleeve will cause further longitudinal expansion forces to be applied to the sleeve, hence causing the sleeve to narrow further and grip the line more tightly. This gives rise to a locking effect. However, it is further noted that release of the line is possible simply by longitudinally compressing the sleeve to cause the diameter of the sleeve to widen and release the line. This longitudinal compression may be achieved by moving at least one of the collars towards the middle of the sleeve. Adjustments to the line may then be made by sliding the line within the sleeve and the line may then be gripped again by releasing the sleeve and allowing the sleeve to return to its expanded configuration.

A fastener according to this invention, an example of which is shown in FIGS. 5a and 5b, comprises an elongate braided tubular sleeve 510 of variable length, and an attachment arrangement for attaching the sleeve to the patient. The attachment arrangement in this case comprises a flange 516 for securing against the skin 528 of a patient. The flange may for example be secured to the skin with a plaster 518 having tear off strips 530 covering an area having an adhesive layer 532. The sleeve 510 may be formed with first and second collars 512, 514 at either end thereof. One of the collars may be attached to the enlarged flange 516 by an arm 520, in a similar manner to the fastener shown in FIGS. 1a and 1b.

A sensor is mounted to the fastener, in this case to the flange 516. The sensor arrangement in this example comprises a sensor element 522, an antenna 524, and a thin battery 526. The sensor may be any required sensor for sensing a physiological parameter of the patient, and/or for sensing a parameter in relation to the line. The sensor may be connected to an antenna for transmitting sensor information, and to a battery for supplying power to the sensor and antenna. The sensor is in this case positioned adjacent the patient's skin 528 when the fastener is attached to the patient. The sensor may for example be a blood pressure or heart rate sensor.

The fastener may additionally or alternatively comprise a line sensor for sensing a condition of a line inserted in the fastener, including a condition of the fluid flow in the line, such as a flow rate sensor, a sensor for detecting an optical property of the fluid, or a sensor for monitoring the length or tension in the sleeve in order to detect the presence of a line therein. Such a sensor may comprise a device connected to the sensor element 522 in the flange 516 via the arm 520. The sensor may thus provide information concerning the integrity of the line, the delivery of the fluid, or the condition of the fluid.

The fastener may communicate with a monitoring device or system for processing the sensor information. The monitoring device may be arranged to store data concerning the patient or the treatment of the patient, such that the device may provide an output, for example an alert signal, if one or more predetermined limits concerning the sensor data are exceeded, indicating a condition which requires the attention of a medical practitioner. For example, the line may have become blocked or detached, or the medical condition of the patient may have deteriorated. Such data may be specific to a particular fastener or type of fastener, or a particular patient or type of patient.

Thus the fastener may also comprise a machine readable marker such as a bar code. A practitioner may programme the monitoring device or system by pairing the bar code with a particular patient, and entering data concerning the patient or sensor parameters associated with the patient. Thus the system may be used to monitor a plurality of fasteners associated with a corresponding plurality of patients.

In addition the fastener may also be associated with a particular practitioner responsible for the care of the patient with which the fastener is associated, for example such that any information or alert corresponding to a particular patient may be sent to a device associated with the practitioner, such a as mobile device which may for example be a mobile phone.

A specific embodiment of a fastener will now be described in more detail with reference to FIGS. 6a, 6b and 7.

The fastener of FIG. 6a includes a braided tubular sleeve 610, having a collar at each end 612, 614. At one end of the sleeve 610, one of the collars 612 is secured to a flange 616. As illustrated in FIG. 1b, the collar 612 is coupled directly to the flange 616. However, in some embodiments the collar 612 is coupled to the flange, for example, via an arm portion. The sensor may be coupled (e.g. mechanically) to sense a parameter relating to the line through this arm portion. The arm portion may be arranged to lift the collar 612 away from the surface of the flange 616, and may comprise an electrical, optical, or acoustic coupling (such as a channel) to mediate sensor data from the line to a controller and/or a data store carried in the flange.

The collar 612 and the flange 616 may be moulded in one piece or may be assembled from separate components. In some embodiments, the collar 612 may be detachably fastenable to the flange 616. For example, the flange 616 may have a clip configured to detachably fasten the collar 612 to the flange, for example the clip may be configured to provide an interference fit between the collar 612 and the clip.

At each end, the braided sleeve 610 passes inside a collar 612, 614. The strands that make up the sleeve 610 are secured to each of the collars 612, 614 using ultrasound welding. Therefore, each strand of the sleeve 610 is securely attached to each collar 612, 614, preventing any fraying or unravelling of the sleeve.

To attach the fastener to a patient, the flange is secured to a plaster 618. The back of the plaster 618 comprises an adhesive material with sufficient adhesion to secure the plaster to a patient's skin. The plaster 618 is provided with backing paper or plastic, which is removed by a medical practitioner when the fastener is in position and the practitioner wishes to secure the fastener to the patient. The fastener may comprise a sensor that is arranged in the flange or foot of the fastener so that, when the plaster is secured to the patient, the sensor is arranged against the patient's skin.

In some embodiments, the flange 616 is secured to the plaster using a second layer of plaster, which is placed over the top of the flange 616 and onto the first plaster layer 618. The second layer of plaster 620 extends over the edges of the flange 616 by about 1 cm in each direction to ensure a secure connection of the flange 616 to the first layer of plaster 618.

As illustrated in FIG. 6a, the flange comprises a sensor 620. The sensor 620 is shown in more detail in FIG. 7. However, it will be understood that other parts of the fastener may comprise the sensor 120, for example the collars 612, 614, the braided tubular sleeve 610 an arm or a clip (if present) may comprise a sensor 620. The fastener may comprise a plurality of sensors for sensing different parameters.

The sensor 620 comprises a sensor element 622 coupled to an antenna 624 and a power source 626, for example a battery. However, in some configurations it will be understood that the sensor 620 need not comprise a battery. For example, information may be obtained from the sensor 620 using RF energy. In such configurations, the sensor 620 may comprise a coupling element for coupling with RF energy.

The sensor element 622 is configured to measure at least one parameter with respect to at least one of the patient, the line and the fastener. For example, the at least one parameter with respect to the line comprises at least one of: fluid flow rate, fluid oxygenation level, fluid temperature, pH level, dissolved gas concentration, age of the line, time since last time the line was replaced, bacteria level. Advantageously, embodiments of the disclosure thus provide an improved fastener that provides an indication of the fluid administered to a patient, for example the quantity or dose administered to a patient, through the line.

For example, the at least one parameter with respect to the fastener comprises at least one of: movement of the fastener, compression in the elongate braided tubular sleeve, tension in the elongate braided tubular sleeve, age of the fastener, time since the fastener was attached to the patient, bacteria level, battery level. Advantageously, embodiments of the disclosure thus provide an improved fastener that provides information regarding the fastener to a healthcare professional, for example whether the line is still attached to the patient. For example, information regarding compression or tension of the elongate braided tubular sleeve may indicate whether the sleeve has been adjusted to replace or remove the line therein.

For example, the at least one parameter with respect to the patient comprises at least one of: blood pressure, heart rate, blood sugar level, blood pH level, blood oxygen level. Advantageously, embodiments of the disclosure thus provide an improved fastener that provides an indication of the health of the patient, for example to a healthcare professional, and may allow the healthcare professional to adjust the flow of fluid in the line in response to the measured parameter.

The sensor 620 is operable to communicate with a remote device, for example remote computer 650, to form a monitoring arrangement. The monitoring arrangement is configured so that the sensor 620 sends an alert signal to computer 650 if at least one parameter falls outside a selected range. For example, an alert signal is triggered if the line has not been replaced for a certain period of time, or if the patient's blood pressure falls below a selected threshold. As another example, an alert signal may be triggered if the patient's body temperature falls below a selected threshold. A significant fall in body temperature may indicate that the fastener has been removed (be it accidentally or deliberately). Advantageously, embodiments of the disclosure provide an improved fastener that provides an indication, for example to a healthcare professional, for example that a line needs replacing, has been removed or has become infected.

The sensor 620 may further be configured to sense other parameters in relation to the patient. For example, the sensor element 622 may be configured to pierce a patient's skin and measure other parameters such as blood glucose level. The monitoring arrangement may thus be configured to respond to the measured parameter, for example blood glucose level, and control the flow of fluid through the line to the patient. For example, if sensor 120 detects a low blood sugar level, the monitoring arrangement may be configured to provide a flow of saline comprising a known concentration of glucose to the patient.

It will of course be understood to the skilled person that such a monitoring arrangement may be used to control the flow of other fluids (liquid or gas) to the patient.

A further example of a fastener is illustrated in FIG. 8, which is a schematic illustration of a giving set, as part of which a fastener may be provided. The giving set includes a tube 812 for supplying fluid, for example saline solution, to a patient. As part of the manufacturing process, a braided tubular sleeve 810 is passed onto the sleeve by compressing the sleeve longitudinally, to widen the braid of the sleeve and sliding the sleeve 810 onto the tube 812.

Connectors (illustrated schematically in FIG. 8) 814, 816 are then attached to each end of the tube 812. It is noted that the connectors 814, 816 are larger than the collars provided at each end of the sleeve 810 and are therefore larger than the maximum diameter to which the sleeve 810 can widen. Therefore, the sleeve cannot pass over the connectors 814, 816 and cannot be removed from the tube 812 once the connectors have been added to each end.

It will be clear to the skilled person that the method and apparatus described above are non-limiting examples and variations may be provided within the scope of the claims.

Apparatus of the disclosure may comprise a plurality of fasteners, and a controller adapted to communicate with each of the fasteners. In these embodiments each fastener may be adapted for securing a medical or surgical line with respect to a patient and comprises a sensor for sensing a physiological parameter of the patient such as their skin temperature or pulse rate. As an alternative, or in addition, each fastener may also comprise a sensor adapted to monitor a parameter relating to the line. Each fastener is coupled to a wireless communications interface, such as a Wi-Fi interface, for reporting the sensed parameter to the controller.

The controller may comprise a processor and may be configured with a rule defining a condition of the sensed parameter. For example, the processor may be configured to perform a selected action based on the sensed parameter. For example the processor may be configured to send an alert to a selected communications device, or to a group of such devices, in the event that the parameter indicates that the line has become dislodged and/or a physiological parameter of the patient has changed in a particular way—e.g. their skin temperature, or pulse is within or outside a selected range or greater than a particular threshold.

The fasteners may comprise a sleeve of variable length for gripping the medical or surgical line. For example the sleeve may be arranged to allow the medical or surgical line to be threaded through it. The sleeve may comprise a braided tubular sleeve arranged so that elongation of the sleeve causes the sleeve to narrow to grip the line and shortening of the sleeve makes the sleeve wider to allow the sleeve to slide along the line, and the parameter in relation to the line is associated with the length of the sleeve.

In these and other embodiments the fasteners may comprise an attachment arrangement for securing the fastener with respect to a patient, and the sensor is arranged so that coupling the attachment arrangement to the patient also arranges the sensor for sensing a physiological parameter of the patient. For example the attachment arrangement may comprise a loop or ring adapted to be sutured to the patient, and the sensor may be disposed on the loop or ring so that when the fastener is in place the sensor is disposed adjacent the patient's skin, for example on the skin. As another example, the attachment arrangement may comprise a foot or flange adapted to lie adjacent the patient's skin and to be held in place with an adhesive plaster or a bandage. The sensor in this case may be coupled to the foot such that securing the fastener to the patient also brings the sensor into contact with the patient's skin for sensing the physiological parameter.

The sensor may comprise a mechanical transducer, for example an acoustic sensor, adapted to sense displacement of the line with respect to the fastener. For example the sensor may be configured to sense elongation and/or shortening of the braid. In these embodiments the attachment means may couple the sensor to the braid—thereby a single sensor or single sensor unit (which may comprise a plurality of sensors for example integrated into a single chip or PCB) may be adapted to sense a physiological parameter (such as heart rate) based on acoustic measurements and also to determine whether the line has become dislodged.

The controllers and processors described herein may comprise any digital logic, such as field programmable gate arrays, FPGA, application specific integrated circuits, ASIC, a digital signal processor, DSP, or by any other appropriate hardware. In addition, all of the methods described herein may be embodied as computer program products operable to program a processor to perform these methods. These computer program products may be carried on non-transitory computer readable storage media and may be distributed as computer readable data carriers, which may include signals transmitted over a network.

Any feature of any one of the examples disclosed herein may be combined with any selected features of any of the other examples described herein. For example, features of methods may be implemented in suitably configured hardware, and the configuration of the specific hardware described herein may be employed in methods implemented using other hardware. In some examples the functionality of the controllers described herein may be provided by a general purpose processor, which may be configured to perform a method according to any one of those described herein.

Where data stores are described it will be appreciated that these may be implemented using memory, such as volatile and/or non-volatile memory.

In some embodiments the fastener comprises an acoustic channel, such as a pipe or tube, adapted to mediate pressure changes from the line and/or from the patient's skin to an acoustic sensor and/or to a temperature sensor. In some embodiments the fastener comprises a recess shaped to enable electronic sensor components to be seated and at least partially encapsulated in the recess. The recess may be arranged so that, when the components are installed in the recess, the sensor is arranged to sense a physiological parameter of the patient when the fastener is, in use, arranged to secure the line with respect to the patient.

At least a part of the fasteners described herein may be manufactured by assembling pre-manufactured components such as plastic fasteners with electronic components. For example, the fasteners may be manufactured by way of an additive manufacture process such as ‘3D printing’ and the electronic components such as the sensor may be installed separately. For example a three-dimensional model of the fastener may be supplied, in machine readable form, to a ‘3D printer’ adapted to manufacture the fastener. This may be by additive means such as extrusion deposition, Electron Beam Freeform Fabrication (EBF), granular materials binding, lamination, photopolymerization, or stereolithography or a combination thereof. The machine readable model comprises a spatial map of the object to be printed, typically in the form of a Cartesian coordinate system defining the object's surfaces. This spatial map may comprise a computer file which may be provided in any one of a number of file conventions. One example of a file convention is a STL (STereoLithography) file which may be in the form of ASCII (American Standard Code for Information Interchange) or binary and specifies areas by way of triangulated surfaces with defined normals and vertices. An alternative file format is AMF (Additive Manufacturing File) which provides the facility to specify the material and texture of each surface as well as allowing for curved triangulated surfaces. The mapping of the apparatus may then be converted into instructions to be executed by 3D printer according to the printing method being used. This may comprise splitting the model into slices (for example, each slice corresponding to an x-y plane, with successive layers building the z dimension) and encoding each slice into a series of instructions. The instructions sent to the 3D printer may comprise Numerical Control (NC) or Computer NC (CNC) instructions, preferably in the form of G-code (also called RS-274), which comprises a series of instructions regarding how the 3D printer should act. The instructions vary depending on the type of 3D printer being used, but in the example of a moving printhead the instructions include: how the printhead should move, when/where to deposit material, the type of material to be deposited, and the flow rate of the deposited material.

The apparatus as described herein may be embodied in one such machine readable model, for example a machine readable map or instructions, for example to enable a physical representation of said apparatus to be produced by 3D printing. This may be in the form of a software code mapping of the apparatus and/or instructions to be supplied to a 3D printer (for example numerical code).

1. A fastener for securing a medical or surgical line with respect to a patient, the fastener comprising: an elongate braided tubular sleeve for receiving the line therethrough, the sleeve having a variable length and being capable when lengthened of gripping the line, an attachment arrangement for attaching the sleeve to the patient, and a sensor mounted to the fastener, the sensor being arranged to sense at least one of a physiological parameter of the patient and a parameter in relation to the line.

2. A fastener as claimed in claim 1, in which the sensor is arranged to be adjacent the skin of the patient when the fastener is attached to the patient, optionally in which the sensor includes a sensor element, a transmitting element and a power source.

3. A fastener as claimed in claim 1 in which the physiological parameter is blood pressure, heart rate, blood sugar level, blood pH level, blood oxygen level, skin translucence, skin conductance, or skin temperature.

4. A fastener as claimed in claim 1 wherein the fastener comprises a sensor configured to measure extension of the elongate braided tubular sleeve, for example wherein the elongate braided tubular sleeve comprises a plurality of braided strands and wherein extension of the sleeve is measured based on at least one of relative angle between at least two of said strands and/or based on compression of at least one strand of the plurality of braided strands and/or compression of the sleeve.

5. The fastener of claim 1 wherein the sensor is configured to provide a monitoring arrangement for the at least one parameter, the sensor being configured to provide an alert signal if the at least one parameter falls outside a selected range, optionally wherein the fastener comprises a first sensor coupling between the patient and the fastener, the first sensor coupling being configured to provide information relating to the patient to sense a parameter in relation to the patient, optionally wherein the fastener comprises a second sensor coupling between parts of the fastener for providing information relating to at least one of the fastener and the line, optionally wherein the monitoring arrangement is configured to provide an alert signal if at least one of the first sensor coupling and the second sensor coupling is severed.

6. A fastener as claimed in claim 1, in which the sensor is arranged to sense a condition in relation to the line through the attachment arrangement, optionally in which the parameter in relation to the fine comprises a condition of a fluid flow in the line, optionally in which the parameter in relation to the line comprises the fluid flow rate, fluid oxygenation level, fluid temperature, pH level, dissolved gas concentration, age of the line, time since last time the line was replaced, or bacteria level.

7. The fastener of claim 1 wherein the sensor is further configured to sense a parameter with respect to the fastener, optionally wherein the at least one parameter with respect to the fastener comprises at least one of movement of the fastener, compression in the elongate braided tubular sleeve, tension in the elongate braided tubular sleeve, age of the fastener, time since the fastener was attached to the patient, bacterial level, and battery level.

8. (canceled)

9. A fastener as claimed in claim 1, in which the sleeve has a first end for fixing adjacent to the patient, in use, and a second end for arranging, in use, proximal to medical equipment coupled to the line, optionally comprising a first substantially rigid collar coupled to the sleeve at the first end of the sleeve, and a second substantially rigid collar coupled to the sleeve at the second end of the sleeve, for varying the length of the sleeve, optionally in which the attachment arrangement comprises an enlarged flange for attaching against the skin of the patient and optionally comprising an adhesive plaster attached to the flange for securing the fastener with respect to the patient, optionally in which the sensor is mounted to the flange, optionally comprising an arm mounting the flange to the collar, the arm providing communication between the sensor and the line.

10. (canceled)

11. (canceled)

12. (canceled)

13. (canceled)

14. (canceled)

15. A system comprising a fastener as claimed in claim 1, and a monitoring arrangement in communication with the sensor and arranged to process data from the sensor, optionally in which the communication is by means of a wireless link, optionally in which the fastener comprises a machine readable marker for pairing with information identifying a patient to whom the fastener is to be secured, optionally in which the monitoring arrangement is arranged to store data representing predetermined sensor parameters relating to a patient and to provide an output according to the relationship between the sensor data and the predetermined parameters, optionally comprising a plurality of fasteners in communication with the monitoring arrangement.

16. (canceled)

17. (canceled)

18. (canceled)

19. (canceled)

20. An apparatus comprising:
a plurality of fasteners adapted for securing a medical or surgical line with respect to a patient, each fastener comprising a wireless communications interface and a sensor for sensing at least one of a physiological parameter of the patient and a parameter in relation to the line;
a controller adapted to communicate with each of the plurality of fasteners via the wireless communications interface, and to trigger an action based on the sensed parameter and a rule defining a condition of the sensed parameter.

21. (canceled)

22. (canceled)

23. (canceled)

24. (canceled)

25. (canceled)

26. The apparatus of claim 20 wherein the fasteners each comprise a unique identifier, and the controller is adapted to store an association between each unique identifier and at least one communications device.

27. The apparatus of claim 26 wherein the controller is adapted to determine whether to send an alert based on a sensed parameter received from a particular fastener and a rule defining a condition of the sensed parameter.

28. The apparatus of claim 27 wherein the controller is configured to select a communications device to which to send the alert based on the unique identifier of the particular fastener and the stored association.

29. The apparatus of claim 20 wherein the fastener, or fasteners, comprise a sleeve of variable length for gripping the line, wherein elongation of the sleeve causes the sleeve to narrow to grip the line and shortening makes the sleeve wider to allow the sleeve to slide along the line, and the parameter in relation to the line is associated with the length of the sleeve.

30. The apparatus of claim 20 wherein the fastener, or fasteners, comprise an attachment arrangement for securing the fastener with respect to a patient, and the sensor is arranged so that coupling the attachment arrangement to the patient also arranges the sensor for sensing a physiological parameter of the patient, optionally wherein the physiological parameter comprises at least one of skin temperature, skin conductivity, pulse rate, and respiratory function.

31. (canceled)

32. The apparatus of claim 20 wherein the sensor comprises a temperature sensor.

33. The apparatus of claim 20 wherein the sensor comprises a mechanical transducer, for example an acoustic sensor, adapted to sense displacement of the line with respect to the fastener.

34. A method of monitoring a plurality of patients in a medical facility, the method comprising:
i. storing a plurality of associations, wherein each association relates one of a plurality of fasteners with a reporting rule and each fastener is adapted for securing a medical or surgical line with respect to the patient and comprises a sensor for monitoring at least one of a physiological parameter of the patient and a parameter relating to the medical or surgical line;
ii. receiving sensor data, via a wireless communications interface, from a particular one of said fasteners;
iii. selecting an action based on the reporting rule associated with the particular one of said fasteners and the sensor data.

35. The method of claim 34 wherein the sensor is adapted to sense movement of the line, or the line becoming unsecured, optionally wherein the fasteners comprise a sleeve of variable length for gripping the line, wherein elongation of the sleeve causes the sleeve to narrow to grip the line and shortening makes the sleeve wider to allow the sleeve to slide along the line, and the parameter relating to the line is associated with the length of the sleeve.

36. (canceled)

37. (canceled)

38. (canceled)

39. (canceled)

40. The method of claim 34 wherein storing the plurality of associations comprises sending the associations, via a wireless communications interface, to a remote device, wherein the remote device is adapted to communicate with the fastener to obtain and monitor the parameter, optionally wherein the action comprises notifying a selected communications device, optionally further comprising obtaining patient identifier data, and selecting the communications device based on the patient identifier, optionally wherein the selected communication device comprises one of a mobile telecommunications device such as a mobile telephone and a selected communications channel such as an email address or wherein the action comprises sending a message to a first communications device, and in the event that an acknowledgement message is not received notifying a second communications device.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)
